(19) United States
(12) Patent Application Publication
(10) Pub. No.: US 2022/0235019 A1
(43) Pub. Date: Jul. 28, 2022

(54) NOVEL IMPROVED METHOD FOR SYNTHESIZING DIAMINOPHENOTHIAZINE COMPOUNDS

(71) Applicant: Achal Narendrakumar AGRAWAL, Udaipur (IN)

(72) Inventor: Achal Narendrakumar AGRAWAL, Udaipur (IN)

(21) Appl. No.: 17/618,339

(22) PCT Filed: Jun. 12, 2020
(86) PCT No.: PCT/IB2020/055519

§ 371 Date: Dec. 10, 2021

(30) Foreign Application Priority Data
Jun. 12, 2019 (IN) 201911009595

(51) Int. Cl. C07D 279/20

(52) U.S. Cl. CPC C07D 279/20

(57) ABSTRACT

The present invention relates to chemical synthesis and purification. Specifically, the present invention relates to a novel and improved method of synthesizing high purity diaminophenothiazine compounds of Formula I, specifically Methylene Blue and its pharmaceutically acceptable salt or hydrates thereof. The present invention relates to an improved method of synthesizing Methylene Blue compound of higher purities than those achievable by using known methods of synthesis as per the requirements of the international pharmacopoeias like USP and EP.

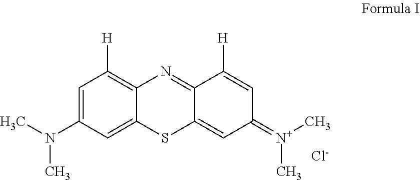

### FIELD OF THE INVENTION

The present invention relates to chemical synthesis and purification. Specifically, the present invention relates to a novel and improved method of synthesizing high purity diaminophenothiazine compounds of Formula I, specifically Methylene Blue and its pharmaceutically acceptable salts or hydrates thereof. The present invention relates to an improved method of synthesizing Methylene Blue compound of higher purities than those achievable by using known methods of synthesis as per the requirements of the international pharmacopoeias like USP and EP.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

The Methylene Blue series (diamino-3,7-phenothiazine) is particularly interesting. This molecule is a common histological dye, less toxic, provided with many biological and pharmacological properties which has a moderate antiseptic action (bacteriostat) and is an antidote to nitrites and methemoglobinizing poisonings. Its photoactivable properties (biocidal singlet oxygen generating product) are already employed as sterilizing agents for blood-derived products. Starting from the 3,7-diaminophenothiazine structure exclusively, a great number of derivatives have been synthesized.

Methylene Blue (Methylthioninium Chloride, MTC), a very well-known diaminophenothiazine dye which is of commercial and medicinal importance can be synthesized using methods well known in the art, involving the synthesis of the zwittterionic indamine compound called Thiosulfonic Acid of Bindschedler's Green and further conversion to Methylene Blue salt by heating the indamine thiosulfonic acid in presence of copper sulphate or other oxidizing agents to temperature equal to or more than 85° C. for elongated periods to complete the conversion. Formation of unwanted dealkylated and degradation related compounds of Methylene Blue such as Azure B, Azure C, Azure A, etc. is very well known for this synthesis route and is mostly reported due to over oxidation by the oxidizing agents such as dichromates, etc used in the manufacturing process.

Methylene Blue (also known as Methylthioninium Chloride), methylthionine chloride; tetramethylthionine chloride; 3,7-bis(dimethylamino) phenazathionium chloride; Swiss blue; C.I. Basic Blue 9; C.I. 52015; is a water soluble, tricyclic organic compound of the following formula:

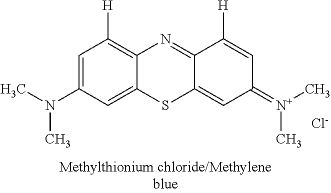

Methylene Blue has molecular formula C₁₆H₁₈ClN₃S (anhydrous) and molecular weight of 319.851 g/mol (anhydrous). Methylene Blue also exists in other salt forms like Zinc Chloride Double salt form and exists in different hydrate forms such as trihydrate, pentahydrate, monohydrate, etc.

Methylene Blue is a very well-known phenothiazine dye and has got various applications like redox indicator, biological stain, anti-dote for cyanide poisoning, treatment for methaemoglobinaemia, etc. In recent years it has been under research for various pharmaceutical applications like treatment of Alzheimer's disease, Dementia, HIV, Parkinson's disease, Cancer Diagnosis, etc.

Methylene Blue synthesis was initially described in German Patent in 1877 (Badische Anilin-und Soda-Fabrik), by nitrosylation of Dimethylaniline, followed by reduction to form N,N-dimethyl-1,4-diaminobenzene and then oxidative coupling in presence of hydrogen sulphide and ferric chloride.

Methods of synthesis of Methylene Blue have also been described by Bernthsen (Bernthsen 1885a, 1885b, 1889). Fierz-David and Bangley, 1949 describes the synthesis of Methylene Blue by treatment of dimethylaniline with sodium nitrite in aqueous hydrochloric acid solution to form p-nitrosodimethylaniline which is further reduced to form p-aminodimethylaniline by using zinc dust in presence of hydrochloric acid. The p-aminodimethylaniline is oxidized using sodium dichromate in aqueous acid solution with another molecule of dimethylaniline and simultaneously a thiosulfonic acid group is introduced to form the indamine Thiosulfonic Acid of Bindschedler's Green. The ring closure is performed by heating the reaction mixture to 85° C. for 30 minutes in presence of manganese dioxide or cupric sulphate to form Methylene Blue. Following reaction sequence has been described in “The Fundamental Processes Of Dye Chemistry”, by Dr. Hans Eduard Fierz-David. The Colour Index (3rd edition of Volume 4, 1971) also describes a very similar synthesis procedure.

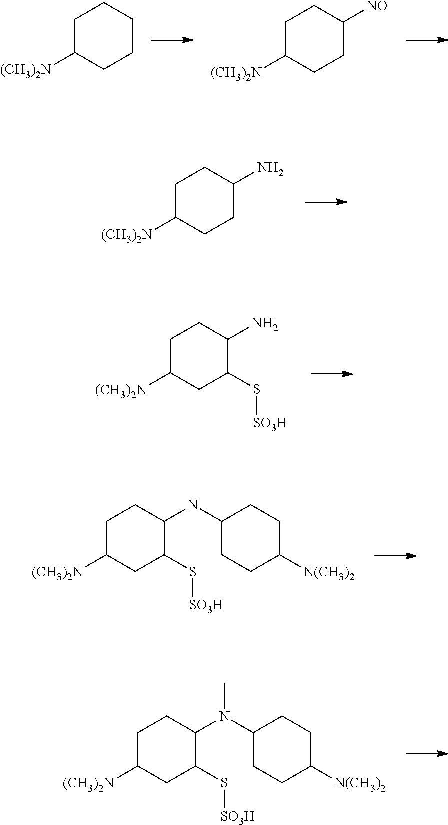

Masuya et al., 1992 also describes routes similar to above for synthesis of certain phenothiazine derivatives.

Marshal and Lewis, 1975, describe the purification process of commercial Methylene Blue by solvent extraction and crystallization to remove Azure B and metal contaminants. They assert that Azure B can be removed from Methylene Blue by extracting a pH 9.5 buffered solution of Methylene Blue containing Azure B, with carbon tetrachloride and performing number of extractions for removal of Azure B.

Lohr et al., 1975, describes the purification of Azure B by column chromatography. It has been described that other cationic dyes such as Methylene Blue can also purified using this method. However, column chromatography is not suitable on large scales.

All the known modifications of the above route of synthesis describe heating of the Thiosulfonic Acid of Bindschedler's Green to temperatures equal to or higher than 85° C. with maintaining of the reaction mixture at temperatures equal to or higher than 85° C. for long durations of time equal to or more than 30 minutes. In a nutshell, all the known modifications of the above described route of synthesis involve the exposure of the Thiosulfonic Acid of Bindschedler's Green and/or formed Methylene Blue to temperatures higher than 60° C. for long durations such as 90 min or more and to temperatures equal to or higher than 85° C. for long durations such as thirty minutes or more.

Formation of degradation impurities mainly Azure B and others like Azure C, Azure A, MVB (Methylene Violet Bernthsen), Thionin, etc. are very well known in the above general route of synthesis and is mainly attributed to the demethylation which occurs during synthesis due to over oxidation by the oxidizing agents used in synthesis such as sodium or potassium dichromate. Unwanted impurity in the form of Azure B with level of more than 5% is mostly reported in the commercial Methylene Blue products. The present European Pharmacopoeia monograph specification of Methylene Blue allows maximum 5% Azure B and other related substances such as Azure A, Azure C, Thionin, etc. with maximum up to 0.1% individually and up to 0.5% in total. The present United States Pharmacopoeia monograph specification of Methylene Blue allows maximum 2.5% Azure B and other related substances such as Azure A, Azure C, Thionin, etc with maximum upto 0.1% individually and up to 0.5% in total. Thus for the pharmaceutical applications, there is requirement of high purity Methylene Blue containing lower levels of degradation impurities, particularly Azure B, Azure A, MVB & Azure C as described in different pharmacopoeias.

U.S. Pat. No. 7,790,881B2 describes isolation and purification of the Zwitterionic Indamine Thiosulfonic Acid of Bindschedler's Green (with optional chromate reduction step) before performing ring closure by heating in presence of acid and copper sulphate. This patent describes further purification steps like solvent extraction, etc. to achieve the pharmacopoeial purity levels. Example No. 1 cited in U.S. Pat. No. 7,790,881B2 describes isolation and purification of the indamine thiosulfonic acid of bindschedler's green and then heating of the same in presence of hydrochloric acid (pH 2) and copper sulfate pentahydrate catalyst, at 85° Celsius for 1 hour after which it is cooled to room temperature. The HPLC analysis results cited by this patent for sample no. CM-pd-378 prepared in accordance to the Example no. 1 shows the Azure B content of 2.89%, MVB content of 0.33% and other impurities content of 0.06%, which is in non-compliance of present USP (United States Pharmacopoeia) and EP (European Pharmacopoeia). In the same patent, sample no CM-pd-378b which was obtained by treatment of sample CM-pd-378 by sodium sulphide and extraction with dichloromethane followed by recrystallization, shows Azure B content of 1.29% and MVB content of 0.14%, passing the requirements of present USP & EP for Azure B but still failing in MVB. Also this patent cites the HPLC analysis results of commercial sample obtained from Medex™ which shows Azure B content of 5.24%, MVB content of 0.1% and 0.44% of other impurities. Table 4 in this patent lists the HPLC analysis results of samples obtained from various sources, all of which show Azure B content higher than 5%. Also the samples DJPS12a and DJPS13a obtained by synthesis and purification according to the claimed methods fail to meet the present criteria of USP and EP for Azure B and other impurities. Further in the same patent, Table 6 lists the HPLC analysis results of 11 samples of Methylene Blue obtained from various well known international sources. Most of the samples show Azure B higher than 5% and even upto 7.52%. Even the reference standard obtained from pharmacopoeia (MTC CRS) shows 3.59% of Azure B content. The Example 2, 4 and 5 of this patent describes heating at 85° C. for 1 hour for ring closure. The Example no. 6 describes addition of isolated and purified Thiosulfonic acid of bindschedler's green to aqueous HCl at ambient conditions, followed by addition of copper sulphate and heating to 85° C. over a period of 15-20 minutes with further stirring at 85° C. for 1 hour and then cooling of reaction mixture to room temperature over a 30 minute period. Example 17 describes heating to 85° C. over 25 minute period (±5 minutes), heating at 85° C. for 60 minutes and cooling back to 60° C. (±2° C.) in period of 20 minutes (±5 minutes).

U.S. Pat. No. 9,242,946B2 granted in 2016 to same assignee as for above mentioned patent, describes similar synthesis methods and claims a Methylene Blue compound with overall purity more than 98% and Azure B content less 2% and/or MVB content less than 0.13%. All the examples of Methylene Blue cited in this patent describe heating upto 85° C. and maintaining the same for 1 hour. It cites only one sample no. CM-pd-378b which has Azure B content less than 2.5% and this level of purity has been achieved by further purification (involving organic extraction) of the sample prepared according to the claimed method.

Another very recent U.S. Pat. No. 10,047,062B2 granted to the same assignee as above and claiming similar process of synthesis with minor modifications cites heating period of 1 hour at 85° C. in the examples. Azure B contents of synthesized samples have not been listed anywhere in the patent. Again granted to the same assignee as above, U.S. Pat. No. 8,691,979B2 describes in all examples, heating at 85° C. for 1 hour for ring closure. Azure B content of any sample has not been cited.

The U.S. Pat. No. 7,956,183B2 describes a method of purification of impure methylene blue containing Azure B and other impurities by acetylation of methylene blue, purification of acetylated compound and then deacetylation to obtain purified methylene blue. Samples obtained after purification of impure starting material containing more than 5% Azure B using the claimed methods are cited, containing Azure B as low as 0.27%. The purity is very high but the purification method involves complex and multistep procedures of acetylation, purification of acetylated compound and deacetylation, recrystallization etc. which consumes lots of costly chemicals and produces lot of effluent and by-products to be discarded. The process is very lengthy, costly and time consuming. In fact it is a purification process and not a synthesis process of Methylene Blue.

U.S. Pat. No. 9,701,648B2 describes method of purification of Methylene Blue by reduction of Methylene Blue using reducing agents like ascorbic acid to form a stable reduced form, isolation and purification of the stabilized reduced form and then oxidation to form Methylene Blue. The minimum content of Azure B after purification is reported as 2.3%. Again, this is a purification process involving multiple steps and substantial amounts of chemicals. This process also generates more amount of effluent and is polluting as well as costly.

The U.S. Pat. No. 8,765,942B2 describes method of synthesis of methylene blue in which an amine substituted derivative compound of methylene blue such as benzoyl leuco-methylene blue is purified and then reacted with a quinone to obtain methylene blue. The above process uses a starting material which is a derivative synthesized from methylene blue itself, hence indirectly involves synthesis of methylene blue and then synthesis of the amine substituted derivative which is further purified and then again reacted to obtain Methylene Blue. This process is very lengthy, consuming lot of chemicals, generating more effluent, polluting and costly. Also the overall yield if calculated taking into consideration the synthesis of initial methylene blue, is very low.

Methylene Blue has got several applications in the field of medicine. Methylene Blue is used as the primary treatment for methemoglobinemia which is a condition in which the methemoglobin levels are high in the blood. Methylene Blue gets reduced to leuco-methyleneblue in blood due to the action of reductase enzyme and the reduced leuco-methylene blue reduces the methemoglobin back to normal haemoglobin, getting oxidized back to methylene blue. Methylene Blue being a redox agent has got therapeutic applications in treating oxidative stress related diseases. Methylene Blue being able to cross the blood brain barrier, has been found useful for treating diseases related to brain and nervous system like Alzheimer's disease, Parkinson's Disease, etc. Oral and parenteral formulations of MTC are commercially available in the United States as urinary antiseptic, usually under the name Urolene Blue®. Methylene Blue is useful in detecting and visualizing cancerous cells and adenomas. Methylene Blue MMX® is under clinical trials for detection and visualization of cancers and adenomas during colonoscopy. Methylene Blue is one of the best known photoantimicrobial and a photodynamic agent in the field of photodynamic therapy (PDT) which is gaining a lot of importance in treating drug resistant viral and bacterial infections and cancers too. Light activated Methylene Blue treatment is already in practice to remove the HIV viral load from the blood plasma. ProvayBlue® 0.5% Methylene Blue injection for treatment of methemoglobinemia has been granted Orphan Drug status in US for treatment of the rare condition methemoglobinemia. However, these formulations contain substantial amounts of metal impurities. These impurities are highly undesirable, and many (e.g., including Al, Cr, Fe, Cu) exceed the safety limits set by European health agencies. It is generally desirable that chemical compounds which are intended to be used as pharmaceuticals are provided in a form that is sufficiently free of undesired impurities. This is especially true for chemical compounds that are intended to be used as part of long-term therapy, for example, daily administration for a period of months or years (or, indeed, indefinitely). The presence of even relatively small amounts of certain undesirable impurities can render a chemical compound unacceptable for use in therapy.

Among the many undesired impurities are certain metals, including especially chromium (Cr). It is often extremely difficult to remove these metal impurities from a chemical compound that has been prepared by a method of chemical synthesis which used them.

Most of the currently available technologies and recently patented processes of Methylene Blue disclose purification processes involving purification of crude methylene blue containing high/unacceptable levels of impurities such as Azure B, Azure C, etc which are difficult to remove by regular purification processes like recrystallization, etc. Most of these purification processes (as disclosed in U.S. Pat. No. 9,382,220B2, U.S. Pat. No. 8,765,942B2, U.S. Pat. No. 8,815,850B2 and U.S. Pat. No. 7,956,183B2) involve derivatization of the crude methylene blue at the phenothiazine ring nitrogen, generally involving reactions like acetylation, acylation, benzoylation, etc which produces derivative of methylene blue like acetyl-leucomethylene blue, benzoyl-leucomethylene blue, etc. resulting in derivative of methylene blue like acetyl-leucomethylene blue, benzoyl-leucomethylene blue, etc. In these reaction sequences, the unwanted impurities like Azure B also get derivatized. The separation of derivatized impurities from the derivatized methylene blue occurs comparatively easily by processes such as filtration, recrystallization, etc. resulting in purified derivatives of methylene blue. The pure derivatized forms of methylene blue are then converted back to methylene blue by de-derivatization, i.e., by removing the chemical entity substituted on the phenothiazine ring nitrogen. However, small quantities of derivatized methylene blue are always detected in the purified methylene blue, thus obtained. Moreover, as such processes involve the use of solvents, residual solvents are also never completely removed. These type of lengthy purification processes involving derivatization and then removal of the derivative (in other words, protection-deprotection strategy), provides pure methylene blue with additional impurity in the form of derivatized methylene blue that could not be completely de-derivatized and always contain more than required amounts of residual solvents.

All the above recent patents describe complex and multistep purification processes post the synthesis of methylene blue and are difficult to perform, employ expensive and toxic reagents, require extreme reaction conditions at industrial scale, are very costly, give loss in yield, and generating more pollutants.

Consequently, there is a need of simpler, shorter, cheaper, scalable, less polluting, environment friendly and easier process of synthesizing Methylene Blue and other phenylthiozinium compounds, that can overcome deficiencies associated with the known arts, and produce products with high purity (pharmaceutical grade purity, i.e., compound having purity safe for human consumption), low or reduced metal content, minimal residual solvent and purity parameters which can meet the limits of degradation impurities mainly Azure B as per the requirements of the international pharmacopoeias like USP and EP.

### OBJECTS OF THE INVENTION

An object of the present invention is to provide an improved method of synthesizing high purity diaminophenothiazine compounds, specifically Methylene Blue, that satisfies the existing needs, as well as others, and generally overcomes the deficiencies found in the prior art.

Another object of the present invention is to provide an improved method of synthesizing high purity Methylene Blue, without the presence of unwanted impurities.

Another object of the present invention is to provide a method of synthesizing high purity Methylene Blue with purity parameters which can meet the limits of degradation impurities mainly Azure B, as per the requirements of the international pharmacopoeias like USP and EP.

The main objective of the present invention is to provide a high purity process for the synthesis of Methylene Blue which is commercially viable and safe on industrial scale.

Another object of the present invention is to provide a method of synthesizing high purity Methylene Blue, as per pharmaceutical grade, i.e., compound having purity safe for human consumption.

Yet another object of the present invention is to provide a method of synthesizing high purity Methylene Blue, which does not involve use of organic solvents.

Another object of the present invention is to provide a method of synthesizing the high purity Methylene Blue that is practically free from the organic solvents, or contains lesser than the allowed residual solvents limits of the different pharmacopoeias.

Another object of the present invention is to provide a method of synthesizing high purity Methylene Blue, which is practically free from the reduced leuco and other substituted forms.

Another object of the present invention is to provide a method of synthesizing high purity Methylene Blue comprising low Azure B impurity content, without the need of isolation and purification of the thiosulfonic acid of bindschedler's green intermediate.

Another object of the present invention is to provide a method of synthesizing high purity Methylene Blue without the need to derivatize methylene blue at the ring nitrogen of phenothiazine ring.

The other objects and preferred embodiments and advantages of the present invention will become more apparent from the following description of the present invention when read in conjunction with the accompanying examples and figures, which are not intended to limit scope of the present invention in any manner.

### SUMMARY OF THE INVENTION

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

The present invention relates to chemical synthesis and purification. Specifically, the present invention relates to a novel and improved method of synthesizing high purity diaminophenothiazine compounds, specifically Methylene Blue of Formula I and its pharmaceutically acceptable salts or hydrates thereof.

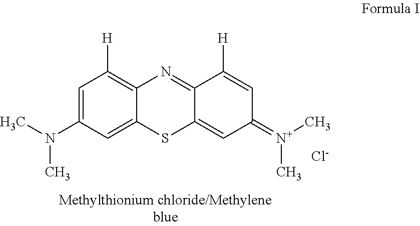

In one aspect, the present invention relates to an improved method of synthesizing high purity Methylene Blue compound, with purity better than those achievable by using known methods of synthesis and as per the requirements of the international pharmacopoeias like USP and EP.

In another aspect, the present invention relates to a method of synthesizing high purity Methylene Blue, as per pharmaceutical grade, i.e., compound having purity safe for human consumption. The present invention provides method of synthesizing Methylene Blue compound with extremely high purity and in particular, products with extremely low levels of undesired impurities.

In yet another aspect, the present invention relates to a method of synthesizing high purity Methylene Blue, with purity parameters which can meet the limits of degradation impurities mainly Azure B as per the requirements of the international pharmacopoeias like USP and EP.

In still another aspect, the present invention relates to a method of synthesizing high purity Methylene Blue, having purities higher than those achievable using known methods of synthesis.

In still another aspect, the present invention relates to a method of synthesizing high purity Methylene Blue, wherein the product is completely free of residual solvents.

In another aspect, the present invention relates to method of synthesizing high purity Methylene Blue compound of Formula I and its pharmaceutically acceptable salts and hydrates thereof, from compounds of Formula II;

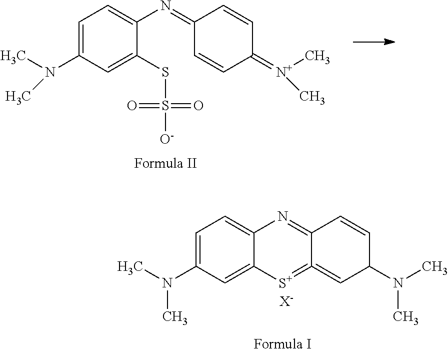

wherein X⁻ represents one or more anionic counter ions to achieve electrical neutrality, preferably chloride.

In another aspect, the present invention relates to a method of synthesizing high purity diaminophenothiazine compound of Formula I, specifically Methylene Blue and its pharmaceutically acceptable salts and hydrates thereof, from compounds of Formula II, comprising the following steps:

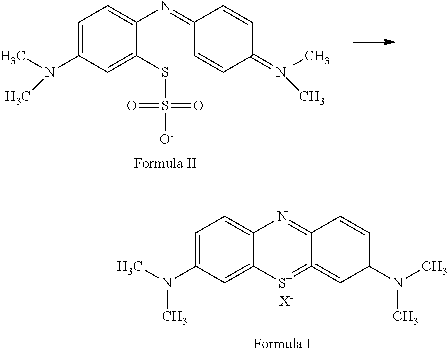

a) Subjecting the zwitterionic indamine compound (ZIC) represented by Formula II, to rapid heating in a liquid medium to achieve a maximum temperature which is at least the temperature required for the thiazine ring closure of the ZIC to form diaminophenothiazine salt of Formula I (DAPS);
b) performing the said thiazine ring cyclization in presence of copper source in catalytic or higher amounts and/or in presence of one or more oxidizing agents;
c) upon achieving the said maximum temperature required for the thiazine ring closure, rapid cooling of the reaction mixture containing the DAPS, so as to allow the exposure of the ZIC and/or the DAPS to elevated temperatures for very short amount of time; and
d) after performing the ring closure, isolating the product formed, DAPS;
   wherein X⁻ represents one or more anionic counter ions to achieve electrical neutrality, preferably chloride and the temperature required for the thiazine ring closure of the ZIC to form diaminophenothiazine salt of Formula I (DAPS) is in the range of 60-105° C.

In one aspect of the present invention, when the anionic counter ion X⁻ is chloride, diaminophenothiazine salt of Formula I (DAPS) is Methylene Blue (also known as Methylthioninium Chloride).

In one aspect, the present invention relates to a method of synthesizing high purity Methylene Blue compound of Formula I and its pharmaceutically acceptable salts and hydrates thereof, wherein high purity Methylene Blue is characterized by:
a) Azure B less than 3% as per European Pharmacopoeia 9.0 HPLC method;
b) Azure A less than 0.1% as per European Pharmacopoeia 9.0 HPLC method;
c) Azure C less than 0.1% as per European Pharmacopoeia 9.0 HPLC method;
d) Sulphated Ash less than 0.25% as per European Pharmacopoeia 9.0 method; and
e) Less than 100 ppm total residual organic solvent content of one or more solvents selected from methanol, ethanol, acetonitrile, acetone, ethyl acetate, dimethylformamide, acetic acid, dichloromethane, carbon tetrachloride, chloroform, cyclohexane, diethyl ether, dimethyl sulfoxide, dichloroethane, 1-propanol, 2-propanol, toluene, tetrahydrofuran or any class 1, class 2 or class 3 solvent, as mentioned in ICH Q3C(R6) guideline and measured by a gas chromatograph with FID detector or a mass detector.

In one aspect, the present invention relates to a method of synthesizing high purity Methylene Blue compound of Formula I and its pharmaceutically acceptable salts and hydrates thereof, wherein pharmaceutically acceptable salts of Methylene Blue can be selected from but not limited to zinc chloride double salt, sulphate salt, nitrate salt, acetate salt, citrate salt, oxalate salt and the like.

In one aspect, the present invention relates to a method of synthesizing high purity Methylene Blue compound of Formula I and its pharmaceutically acceptable salts and hydrates thereof, wherein pharmaceutically acceptable hydrates of Methylene Blue can be selected from but not limited to monohydrate, dihydrate, trihydrate, tetrahydrate, pentahydrate, heptahydrate and hexahydrate.

In one aspect, the present invention relates to a method of synthesizing high purity Methylene Blue compound of Formula I, wherein high purity Methylene Blue can be further derivatized into reduced and stable forms like acetyl leuco-Methylene Blue, Benzoyl Leuco-methylene Blue, Leco-methylene Blue Dihydrochloride, Leuco-methylene Blue Mesylate, Leuco-methylene Blue Ascorbate and the like.

In one aspect, the present invention relates to pharmaceutical compositions comprising high purity Methylene Blue compound of Formula I.

In another aspect, the present invention relates to preparation of medicament comprising high purity Methylene Blue compound of Formula I.

In another aspect, the present invention relates to use of high purity Methylene Blue compound of Formula I, for the preparation of medicament.

Various objects, features, aspects and advantages of the inventive subject matter will become more apparent from the following detailed description of preferred embodiment.

### DETAILED DESCRIPTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Various terms as used herein are shown below. To the extent a term used in a claim is not defined below, it should be given the broadest definition persons in the pertinent art have given that term as reflected in printed publications and issued patents at the time of filing.

The present invention relates to chemical synthesis and purification. Specifically, the present invention relates to a novel and improved method of synthesizing high purity diaminophenothiazine compounds, specifically Methylene Blue of Formula I and its pharmaceutically acceptable salts or hydrates thereof.

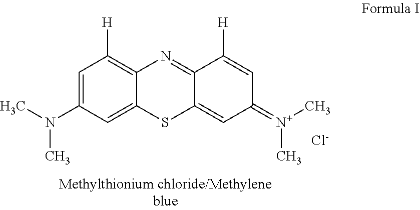

In another embodiment, the present invention relates to a novel and improved method of synthesizing Methylene Blue compound of high purity, as per pharmaceutical grade, i.e., compound having purity safe for human consumption. The present invention provides method of synthesizing Methylene Blue compound with extremely high purity and in particular, products with extremely low levels of undesired impurities.

In one embodiment, the present invention relates to an improved method of synthesizing higher purities Methylene Blue compound with purities higher than those achievable by using known methods of synthesis and as per the requirements of the international pharmacopoeias like USP and EP.

The present European Pharmacopoeia monograph specification of Methylene Blue allows maximum 5% Azure B and other related substances such as Azure A, Azure C, Thionin, MVB etc. with maximum up to 0.1% individually and up to 0.5% in total. The present United States Pharmacopoeia monograph specification of Methylene Blue allows maximum 2.5% Azure B and other related substances such as Azure A, Azure C, Thionin, MVB etc with maximum upto 0.1% individually and up to 0.5% in total. In the commercially available Methylene Blue products, unwanted impurity in the form of Azure B with level of more than 5% is mostly reported.

Other commercially available formulations of Methylene Blue products contain substantial amounts of metal impurities. These impurities are highly undesirable, and many (e.g., including Al, Cr, Fe, Cu) exceed the safety limits set by European health agencies. The chemical compounds which are intended to be used as pharmaceuticals require high purity substrates that are substantially free of undesired impurities.

In yet another embodiment, the present invention relates to an improved method of synthesizing high purity Methylene Blue, with purity parameters which can meet the limits of degradation impurities mainly Azure B as per the requirements of the international pharmacopoeias like USP and EP.

Most of the currently available technologies and recently patented processes of Methylene Blue disclose purification processes involving purification of crude methylene blue containing high/unacceptable levels of impurities such as Azure B, Azure C, etc which are difficult to remove by regular purification processes like recrystallization, etc. Most of these purification processes involve derivatization of the crude methylene blue at the phenothiazine ring nitrogen, generally involving reactions like acetylation, acylation, benzoylation, etc which produces derivative of methylene blue like acetyl-leucomethylene blue, benzoyl-leucomethylene blue, etc. resulting in derivative of methylene blue like acetyl-leucomethylene blue, benzoyl-leucomethylene blue, etc. In these reaction sequences, the unwanted impurities like Azure B also get derivatized. The separation of derivatized impurities from the derivatized methylene blue occurs comparatively easily by processes such as filtration, recrystallization, etc. resulting in purified derivatives of methylene blue. The pure derivatized forms of methylene blue are then converted back to methylene blue by de-derivatization, i.e., by removing the chemical entity substituted on the phenothiazine ring nitrogen. However, small quantities of derivatized methylene blue are always detected in the purified methylene blue, thus obtained. Moreover, as such processes involve the use of solvents, residual solvents are also never completely removed. These type of lengthy purification processes involving derivatization and then removal of the derivative (in other words, protection-deprotection strategy), provides pure methylene blue with additional impurity in the form of derivatized methylene blue that could not be completely de-derivatized and always contain more than required amounts of residual solvents.

The high purity methylene blue prepared according to the embodiments of the present invention, provides high purity methylene blue without the need for such lengthy processes. Moreover, since the present method does not require purification by derivatization, the high purity methylene blue of the present invention is completely free from derivatized impurities. Additionally, since no solvent is used at the cyclization step or the purification process, there is absolutely no residual solvent in the high purity methylene blue prepared according to the embodiments of the present invention.

The ICHQ3C(R6) guideline adopted by most of the countries and international pharmacopoeias, describes residual organic solvents as class 1, class 2 and class 3 solvents and specifies limits of many of the organic solvents in the active substances and drug products. There have been several reports wherein the presence of residual organic solvents was found to be the reason for contamination of active substances with carcinogenic nitrosamines. Most of the currently available processes for synthesis and purification involve the use of organic solvents; hence the presence of residual amounts of organic solvents in the product Methylene Blue active substance prepared from those processes is quite inevitable. All the currently known processes for purification of Methylene blue from unwanted impurity Azure B involve the use of organic solvent. The residual organic solvents even if present in the drug substance or product within allowable limits makes the product unfit for its therapeutic value.

The present invention provides a novel and inventive process for the preparation of high purity methylene blue, wherein presence of impurities like Azure C, MVB, thionine etc., can be avoided and the process results in a product with absolutely no residual solvent as no organic solvent is utilized in either the cyclization step leading to formation of methylene blue or in its purification.

In another embodiment, the present invention relates to method of synthesizing high purity diaminophenothiazine salt of Formula I, specifically Methylene Blue and its pharmaceutically acceptable salts and hydrates thereof, from compounds of Formula II,

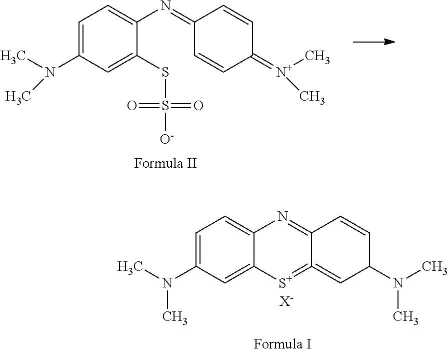

Wherein X⁻ represents one or more anionic counter ions to achieve electrical neutrality, preferably chloride.

In another embodiment, the present invention relates to a method of synthesizing high purity Methylene Blue compound of Formula I and its pharmaceutically acceptable salts and hydrates thereof, from compounds of Formula II, comprising the following steps:

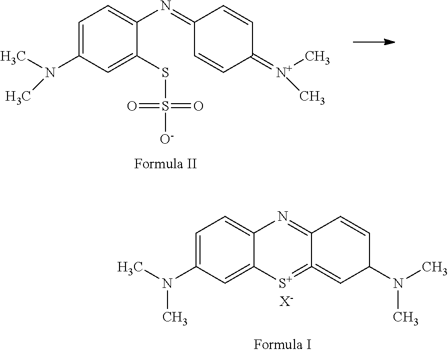

a) Subjecting the zwitterionic indamine compound (ZIC) represented by Formula II, to rapid heating in a liquid medium to achieve a maximum temperature which is at least the temperature required for the thiazine ring closure of the ZIC to form diaminophenothiazine salt of Formula I (DAPS);
b) performing the said thiazine ring cyclization in presence of copper source in catalytic or higher amounts and/or in presence of one or more oxidizing agents;
c) upon achieving the said maximum temperature required for the thiazine ring closure, rapid cooling of the reaction mixture containing the DAPS, so as to allow the exposure of the ZIC and/or the DAPS to elevated temperatures for very short amount of time; and
d) after performing the ring closure, isolating the product formed, DAPS; wherein X⁻ represents one or more anionic counter ions to achieve electrical neutrality, preferably chloride and the temperature required for the thiazine ring closure of the ZIC to form diaminophenothiazine salt of Formula I (DAPS) is in the range of 60-105° C.

In one aspect of the present invention, when the anionic counter ion X⁻ is chloride, diaminophenothiazine salt of Formula I (DAPS) is Methylene Blue (also known as Methylthioninium Chloride).

In one embodiment, the present invention relates to a method of synthesizing high purity Methylene Blue compound of Formula I and its pharmaceutically acceptable salts and hydrates thereof, wherein high purity Methylene Blue is characterized by:
a) Azure B less than 3% as per European Pharmacopoeia 9.0 HPLC method;
b) Azure A less than 0.1% as per European Pharmacopoeia 9.0 HPLC method;
c) Azure C less than 0.1% as per European Pharmacopoeia 9.0 HPLC method;
d) Sulphated Ash less than 0.25% as per European Pharmacopoeia 9.0 method; and
e) Less than 100 PPM of total residual organic solvent content of one or more solvents selected from methanol, ethanol, acetonitrile, acetone, ethyl acetate, dimethylformamide, acetic acid, dichloromethane, carbon tetrachloride, chloroform, cyclohexane, diethyl ether, dimethyl sulfoxide, dichloroethane, 1-propanol, 2-propanol, toluene, tetrahydrofuran or any class 1, class 2 or class 3 solvent, as mentioned in ICH Q3C(R6) guideline and measured by a gas chromatograph with FID detector or a mass detector.

In a preferred embodiment, the present invention relates to a method of synthesizing high purity Methylene Blue compound of Formula I and its pharmaceutically acceptable salts and hydrates thereof, wherein content of Azure B in high purity Methylene blue is less than 2.5%, preferably less than 2.0%, more preferably less than 1.8% as per European Pharmacopoeia 9.0 HPLC method.

In a preferred embodiment, the present invention relates to a method of synthesizing high purity Methylene Blue compound of Formula I and its pharmaceutically acceptable salts and hydrates thereof, wherein content of Azure A in high purity Methylene blue is less than 0.05%, and content of Azure C is less than 0.05%, as per European Pharmacopoeia 9.0 HPLC method.

In one embodiment, the present invention relates to a method of synthesizing high purity Methylene Blue compound of Formula I and its pharmaceutically acceptable salts and hydrates thereof, wherein total residual organic solvent content of one or more solvents in high purity Methylene blue is less than 50 ppm, preferably less than 10 ppm, more preferably less than 5 ppm, highly preferably less than 1 ppm and most preferably less than 0.1 ppm.

In one embodiment, the present invention relates to a method of synthesizing high purity Methylene Blue compound of Formula I and its pharmaceutically acceptable salts and hydrates thereof, wherein content of total elemental impurities in high purity Methylene Blue is less than the amount allowable according to limits of European Pharmacopoeia 9.0.

In a preferred embodiment, the present invention relates to a method of synthesizing high purity Methylene Blue compound of Formula I and its pharmaceutically acceptable salts and hydrates thereof, wherein content of total elemental impurities in high purity Methylene Blue is less than 0.75 times the limit prescribed in the European Pharmacopoeia 9.0, preferably less than 0.5 times the limit prescribed in the European Pharmacopoeia 9.0.

In one embodiment, the present invention relates to a method of synthesizing high purity Methylene Blue compound of Formula I and its pharmaceutically acceptable salts and hydrates thereof, wherein content of MVB is less than 0.05% and content of Thionin is less than 0.05%.

In one embodiment, the present invention relates to a method of synthesizing high purity Methylene Blue compound of Formula I and its pharmaceutically acceptable salts and hydrates thereof, wherein any diaminophenothiazine compound other than methylene blue and Azure B is not present more than 0.05%.

The limits of total elemental impurities allowable according to limits of Pharmacopoeia are given in the below table:

| | Limit as per Ph. Eur. |
|---|---|
| Element Name | and USP (ppm) |
| Arsenic* | 8 |
| Zinc | 100 |
| Copper* | 200 |
| Aluminum | 100 |
| Cadmium | 1 |
| Chromium | 100 |
| Tin | 10 |
| Iron | 200 |
| Manganese | 10 |
| Mercury | 1 |
| Molybdenum | 10 |
| Nickel | 10 |
| Lead | 10 |

*Limit of Arsenic and copper are as per USP-42 monograph and rest all are as per Ph Eur 9.0 monograph.

The thiosulfonic acid of bindschedler's green (also referred to as ZIC, represented by Formula II), is an intermediate in the synthesis of Methylene Blue and there is no other known use of this compound. ZIC is synthesized by oxidative coupling of N,N-Dimethylaniline with 2-Amino-5-Dimethylaminophenyl Thiosulfonic acid. Generally, during the synthesis of Methylene Blue, ZIC is not isolated and is further subjected to ring closure in-situ to form Methylene Blue. Only one US patent (U.S. Pat. No. 7,790,881) describes oxidative coupling of N,N-Dimethylaniline with 2-Amino-5-Dimethylaminophenyl Thiosulfonic acid involving Cr(VI), isolation and purification of the Thiosulfonic Acid of Bindschedler's Green, ring closure of purified Thiosulfonic Acid of Bindschedler's Green using copper sulphate as catalyst in acidic aqueous medium to form methylene blue.

According to embodiments of the present invention, ZIC compound of Formula II, used for the synthesis of compound of Formula I, is prepared by oxidative coupling of N,N-Dimethylaniline with 2-Amino-5-Dimethylaminophenyl Thiosulfonic acid and is used further for cyclization reaction, without isolation or purification. The oxidative coupling of ZIC is usually performed in temperature conditions between 0-20° C., preferably below 5° C.

When the ring closure/cyclization is performed without isolation of the Thiosulfonic Acid of Bindschedler's Green (ZIC), the reaction mixture comprising ZIC, which is below or at room temperature, is heated to elevated temperatures between 80 to 100° C. and heated for considerable time, in presence of oxidizing agents in catalytic or higher amounts, to achieve the ring closure resulting in the formation of methylene blue.

When the ring closure is performed on an isolated Thiosulfonic Acid of Bindschedler's Green, the Thiosulfonic Acid of Bindschedler's Green is added to an acidic aqueous medium below or at room temperature or usually below 60° C., heated to elevated temperatures between 80 to 100° C. and heated for considerable time, in presence of oxidizing agents in catalytic or higher amounts, to achieve the ring closure and formation of methylene blue.

All of the known processes involve a considerable time of exposure, of ZIC and/or the ring closed methylene blue compound, to temperatures higher than 60° C., during the process of heating to achieve the ring closure temperature, during the ring closure reaction at elevated temperatures between 70-100° C. and during the cooling back of the reaction mixture to normal conditions or to temperature below 60° C.

None of the known process discusses the degradation of the ZIC and/or the ring closed Methylene Blue compound, due to elongated exposure to the temperatures higher than 60° C. Most of the known processes for manufacturing pure Methylene Blue have focused on purification of a crude methylene blue with high level of degradation impurities by using lengthy, time consuming, non-ecofriendly and costly processes.

In one embodiment, the present invention relates to a method of synthesizing high purity Methylene Blue of Formula I, wherein the ZIC compound of Formula II is added to the hot liquid medium which is at temperature of 60 to 105° C.

In one embodiment, the present invention relates to method of synthesizing high purity Methylene Blue of Formula I, wherein the temperature required for the thiazine ring closure of the ZIC to form diaminophenothiazine salt of Formula I (DAPS) is in the range of 60-105° C., preferably 70-95° C.

In one embodiment, the present invention relates to a method of synthesizing high purity Methylene Blue of Formula I, wherein the ZIC compound of Formula II is subjected to the rapid heating required for the ring closure, for very short period of time, such that the rise of temperature from 60° C. to 78° C. is achieved in 5 to 15 minutes.

In another embodiment, the present invention relates to a method of synthesizing high purity Methylene Blue of Formula I, wherein the cyclized DAPS of Formula I, is exposed to elevated temperatures for very short period of time.

In another embodiment of the present invention, quick ring closure leading to formation of cyclized diaminophenothiazinium salt of Formula I is such that the exposure of cyclized diaminophenothiazinium to temperatures in the range of 78-100° C. is for 2 to 45 minutes, preferably 2 to 30 minutes, more preferably 2 to 15 minutes, highly preferably 2 to 10 minutes and most preferably 2 to 5 minutes.

In yet another embodiment, the present invention relates to a method of synthesizing high purity Methylene Blue of Formula I, wherein ring closure reaction is carried out by rapidly heating the reaction mixture containing the Zwitterionic Indamine compound of Formula II, to the ring closure temperature and rapid cooling back of the reaction mixture containing the resulting Diaminophenothiazine salt of Formula I, characterized in that the rapid cooling is carried out such that the temperature is brought down from 78° C. to 60° C. in 2 to 15 minutes.

In a preferred embodiment of the present invention, thiazine ring cyclization is performed by subjecting the ZIC to rapid heating in such a manner that rise in temperature from 60° C. to temperature between 75° C. to 95° C. is achieved in 2 to 15 minutes and again rapid cooling back of the reaction mixture containing the formed diaminophenothiazinium compound to temperature below 60° C. in 5 to 30 minutes of achieving the maximum temperature between 75° C. to 95° C., not allowing the temperature to remain at or above 75° C. for more than 15 minutes.

The important difference between the known methods and the presently claimed method of synthesis is that the present invention utilizes rapid heating and rapid cooling of the reaction mixture and exposing the product formed to higher temperatures for a minimum period of time, resulting in very less amount of unwanted impurities. According to the present invention, the ZIC is heated rapidly to temperatures in the range of 75° C. to 95° C. for the thiazine ring closure and again rapidly cooled back to temperatures less than 60° C. immediately upon achieving the desired maximum temperature between 75° C. to 95° C., not keeping the reaction mixture above 75° C. for more than 15 minutes wherein performing of the rapid heating in such a manner that rise in temperature from 60° C. till the maximum temperature is done within 15 minutes and rapid cooling from the maximum achieved temperature to 60° C. is done in less than 30 minutes.

According to one embodiment of the present invention, the method of synthesizing high purity Methylene Blue not only gives the Methylene Blue compound in sufficient and good yields but also gives a high purity Methylene Blue compound with the content of Azure B and other related compounds in full compliance of the present requirements of the monographs of USP and EP pharmacopoeias and even better than that, without any further purification required in particularly for removal of Azure B and other related contaminants such as Azure A, Azure C, MVB, Thionin, etc.

In one embodiment of the present invention, the reaction mixture should not be exposed to temperature in the range of 60-105° C. for more than 60 minutes including the time taken for heating above 60° C. and the time taken for cooling till 60° C.

In a preferred embodiment of the present invention, the reaction mixture is exposed to temperatures in the range of 60-105° C. for 2 to 60 minutes, preferably 2 to 45 minutes, more preferably 2 to 30 minutes and most preferably 2 to 15 minutes.

In another embodiment of the present invention, the reaction mixture is exposed to temperature in the range of 78-105° C. for 1 to 45 minutes, preferably 1 to 30 minutes, more preferably 1 to 20 minutes and most preferably 1 to 10 minutes.

In one embodiment, the present invention relates to a method of synthesizing high purity Methylene Blue of Formula I, wherein the copper source used for thiazine ring cyclization can be selected from but not limited to copper sulphate, copper compound, copper ions and the like.

In one embodiment the catalyst used is copper source, preferably Copper(II) Sulfate, in quantities ranging from (on anhydrous basis) 0.05 mol per mole of ZIC to 0.5 mol per mole of ZIC.

In a preferred embodiment, the catalyst used is Copper(II) Sulfate in quantities ranging from (on anhydrous basis) 0.1 mol per mole of ZIC to 0.15 mol per mole of ZIC.

In another preferred embodiment, the copper sulphate is used in pentahydrate form.

In a preferred embodiment, the rapid heating of acidic water at pH 2-3, containing ZIC and copper catalyst is carried out at 90° C. and after 5 minutes the rapid cooling is started and then within 10 minutes temperature of 60° C. is achieved.

In one embodiment, the rapid heating of acidic water at pH 2-3, containing ZIC and copper catalyst is carried out at 95° C. and after 2 minutes the rapid cooling is started and then within 10 minutes temperature of 60° C. is achieved.

In one embodiment, the rapid heating of acidic water at pH 2-3, containing ZIC and copper catalyst is carried out at 95° C. and after and after 2 minutes the rapid cooling is started and then within 5 minutes temperature of 60° C. is achieved.

In one embodiment the rapid heating is done by supplying the reaction mixture below 30° C. to a tubular continuous flow type reactor/heat exchanger (externally heated) to achieve the maximum temperature of 85° C. within 1 minute and then not keeping the reaction mixture at or above 85° C. for more than a minute, the reaction mixture is supplied to another tubular continuous flow type reactor/heat exchanger (externally cooled) to achieve temperature of 50° C. within 1 minute.

Surprisingly and unexpectedly, it has been observed that when the zwitterionic indamine thiosulfonic acid compound of Formula II is subjected to the necessary heating required for the ring closure, for very short period of time and also if the formed diaminophenothiazinium salt of Formula I is exposed to elevated temperatures for very short period of time, rather than the elongated periods as described in various known arts and processes, the dealkylation or degradation of the principle compound is very less as compared to the dealkylation or degradation which occurs when the process is carried out according to the known method.

More specifically, if the reaction mixture is exposed to the temperatures higher than 60° C. for very short amount of time, then very less amount of dealkylation occurs. In order to perform the ring closure reaction in a manner exposing the reaction mixture to higher temperatures for short period of time, rapid heating of the reaction mixture containing the ZIC of Formula II to the ring closure temperature and rapid cooling back of the reaction mixture containing the formed DAPS of Formula II is the best way. Consequently, the product of Formula I is obtained in high purity.

The inventive ingenuity of the inventors of the present invention lie in the fact that inventors have determined through rigorous experimentation that unwanted impurity in the form of Azure B is generated during the synthesis not only due to the over oxidation by the used oxidants but also due to the temperatures higher than 60° C. and further higher temperatures of 75° C. and more, to which the ZIC and most importantly the Methylene Blue is exposed for longer periods which is surprisingly not required for an efficient ring closure to happen and rather, exposing to such temperatures for longer periods causes degradation. This important fact has not been disclosed in any prior art literature including very recently filed and granted patents. Rapid heating and rapid cooling of the reaction mixture is the most essential parameter of the process for obtaining high purity Methylene Blue, and solves the technical problem of obtaining high purity methylene blue that meets the requirements of the international pharmacopoeias like USP and EP.

In another embodiment, the present invention relates to a method of synthesizing high purity diaminophenothiazinium compounds, specifically Methylene blue of Formula I, which does not involve use of organic solvents selected from but not limited to methanol, ethanol, acetonitrile, acetone, ethyl acetate, dimethylformamide, acetic acid, dichloromethane, carbon tetrachloride, chloroform, cyclohexane, diethyl ether, dimethyl sulfoxide, dichloroethane, 1-propanol, 2-propanol, toluene, tetrahydrofuran or any class 1, class 2 or class 3 solvent, as mentioned in ICH Q3C(R6) guidelines.

In yet another embodiment, the present invention relates to a method of synthesizing high purity diaminophenothiazinium compounds, specifically Methylene blue of Formula I, which are practically free from the organic solvents selected from but not limited to methanol, ethanol, acetonitrile, acetone, ethyl acetate, dimethylformamide, acetic acid, dichloromethane, carbon tetrachloride, chloroform, cyclohexane, diethyl ether, dimethyl sulfoxide, dichloroethane, 1-propanol, 2-propanol, toluene, tetrahydrofuran or any class 1, class 2 or class 3 solvent, as mentioned in ICH Q3C(R6) guidelines, or contain lesser than the allowed residual solvents limits of the different pharmacopoeias like USP, EP, etc.

In one embodiment, the present invention relates to an improved method of synthesizing diaminophenothiazinium compounds of Formula I wherein at any point of time between the rapid heating and quick ring closure, ZIC is partially or fully converted into a thiol or a disulfide.

In another embodiment, the present invention relates to an improved method of synthesizing high purity Methylene Blue of Formula I, wherein the oxidizing agent used during cyclization step can be selected from manganese dioxide, sodium dichromate, persulfates, and the like.

In yet another embodiment, the present invention relates to an improved method of synthesizing high purity Methylene Blue of Formula I, wherein the isolation step of DAPS may further involve additional steps including, but not limited to filtration, pH adjustment, acidification, salt formation, crystallization, cooling, salting out, precipitation and the like.

In one embodiment of the present invention, one or more treatment steps can be optionally applied after the ring closure and before the isolation of DAPS, like pH adjustment, alkalization, etc. in order to remove inorganic and other impurities.

In one embodiment, the filtration or one or more treatment steps are performed after the ring closure and before the rapid cooling.

In one embodiment, the filtration or one or more treatment steps are carried out after the ring closure and during the rapid cooling.

In one embodiment, the present invention relates to a method of synthesizing high purity Methylene Blue of Formula I, wherein the ZIC compound is in reduced state.

In one embodiment, the present invention relates to a method of synthesizing high purity Methylene Blue of Formula I, wherein ZIC compound is in form of alkaline metal salt or any other salt form.

In another embodiment of the present invention, thiazine ring cyclization is performed in a liquid medium wherein the liquid medium is acidic aqueous medium, which is either water or mixture of water and other polar solvents like methanol, acetonitrile, acetone etc., under acidic pH conditions.

In one embodiment, the reaction medium is acidified water which is acidified with one or more acid comprising hydrochloric acid, sulphuric acid, acetic acid, etc.

In one embodiment, the pH of reaction medium is acidic and is in the range of 0.5 to 6.5, preferably 1 to 5, more preferably 2 to 4 and most preferably 2 to 3.

In one embodiment the acid used for acidification of the reaction medium is selected from but not limited to hydrochloric acid, sulphuric acid, acetic acid and the like.

In one embodiment, the present invention relates to a method of synthesizing high purity Methylene Blue compound of Formula I and its pharmaceutically acceptable salts and hydrates thereof, wherein pharmaceutically acceptable salts of Methylene Blue can be selected from but not limited to zinc chloride double salt, sulphate salt, nitrate salt, acetate salt, citrate salt, oxalate salt and the like. Methylene Blue is most widely used in the chloride salt form but other salt forms of Methylene Blue are also widely used in industry. Zinc Chloride Double salt of Methylene Blue is widely used form in paper and textile dye. The citrate salt of Methylene Blue is a highly preferred form for preparing pharmaceutical compositions with silver ions as silver ions are non-compatible with chloride ions. Methylene Blue cation can also bond with the anions of anionic surfactants such as sulfonic acid salts (like Sodium Lauryl Ether Sulfate), alcohol sulfates, alkylbenzene sulfonates, phosphoric acid esters, and carboxylic acid salts. Methylene Blue cation bonded with the anion of anionic surfactant can be isolated using well known methods like solvent extraction, etc.

In one embodiment, the present invention relates to a method of synthesizing high purity Methylene Blue compound of Formula I and its pharmaceutically acceptable salts and hydrates thereof, wherein pharmaceutically acceptable hydrates of Methylene Blue can be selected from but not limited to monohydrate, dihydrate, trihydrate, tetrahydrate, pentahydrate, hexahydrate and heptahydrate.

In one embodiment, the synthesized DAPS, specifically methylene blue of Formula I, is isolated by salt formation.

In another embodiment, DAPS is isolated as corresponding chloride salt or zinc chloride salt.

In a preferred embodiment, the salt formation of product formed, DAPS, is carried out after the ring closure, by treatment with hydrochloric acid, zinc chloride or any other chloride salt.

In one embodiment the Zinc Chloride Double salt of Methylene Blue is precipitated by adding zinc chloride to the aqueous solution containing methylene blue.

In one embodiment the nitrate salt of Methylene Blue is prepared by adding Silver Nitrate to the aqueous solution of Methylene Blue, wherein Silver Chloride precipitates leaving Methylene Blue Nitrate salt in the solution.

In one embodiment, the DAPS formed after the ring closure is isolated after its synthesis from the reaction mixture in which it was synthesized and optionally purified.

In one embodiment, the DAPS formed after the ring closure is not isolated and is used further for synthesis of its derivatives, in the reaction mixture containing the DAPS.

In one embodiment, the present invention relates to a method of synthesizing high purity Methylene Blue compound of Formula I, wherein high purity Methylene Blue can be further derivatized into reduced and stable forms like acetyl leuco-Methylene Blue, Benzoyl Leuco-methylene Blue, Leuco-methylene Blue Dihydrochloride, Leuco-methylene Blue Mesylate, Leuco-methylene Blue Ascorbate and the like.

In one embodiment, the DAPS formed after the ring closure is reduced to its leuco form using reducing agents selected from but not limited to ascorbic acid, sodium dithionite and sodium borohydride.

In one embodiment of the present invention, the acidic aqueous solution containing methylene blue is reduced using ascorbic acid and the stable leuco-methylene blue ascorbate is isolated by filtration.

In one embodiment, the DAPS formed after the ring closure is reduced to its leuco form before or after applying any of the treatment steps.

In one embodiment, the DAPS formed after the ring closure is reduced to its leuco form and isolated.

In one embodiment, the DAPS formed after the ring closure is reduced to its leuco form and used without isolation for formation of derivatives.

In another embodiment of the present invention, an aqueous solution containing methylene blue is reduced using sodium dithionite and then benzoylated using benzoyl chloride to form benzoyl leucomethylene blue.

In still another embodiment of the present invention, the aqueous solution containing methylene blue is reduced using dithionite and the stable leucomethylene blue dihydrochloride is isolated using hydrochloric acid.

In yet another embodiment of the present invention, the aqueous solution containing methylene blue is reduced using dithionite and the stable leucomethylene blue mesylate is isolated using methanesulfonic acid.

In another embodiment, the present invention provides the shortest, fastest, easiest, cheapest, less polluting and environment friendly process for synthesis of high purity of diphenylthiazine compounds, preferably methylene blue compound which is in compliance to the requirements of various present international pharmacopoeias like USP and EP, for the related compounds/contaminants such as Azure B, Azure A, Azure C, MVB, etc.

In another embodiment of the present invention, thiazine ring cyclization is performed using Zwitterionic Indamine Compound (ZIC) of Formula II, wherein the ZIC is used with or without isolation/purification of the same from the reaction mixture in which it is formed.

In one embodiment the ZIC is generated in situ by oxidative coupling of thiosulfonic acid of p-aminodimethylaniline with dimethylaniline using oxidizing agent such as sodium/potassium dichromate, manganese dioxide etc. in acidic medium comprising water or mixture of water and one or more water miscible solvent like methanol, acetonitrile, etc.

In another embodiment the ZIC generated by oxidative coupling of thiosulfonic acid of p-aminodimethylaniline with dimethylaniline using oxidizing agent such as manganese dioxide, etc in an acidic medium comprising of water or mixture of water and one or more water miscible solvent like methanol, acetonitrile, etc, is isolated and optionally purified.

In yet another embodiment, ZIC synthesized by any available route of synthesis is used for further reaction according to the present invention.

In one embodiment, the rapid heating is performed by supplying indirect heat using steam, hot oil, electric heater, etc.

In one embodiment, the rapid heating is performed by supplying live steam directly.

In one embodiment, the rapid heating is done by adding the reaction mixture containing the ZIC to a hot reaction vessel.

In one embodiment the rapid heating is done by microwave irradiation.

In one embodiment the rapid heating is done by passing the reaction mixture below 60° C. through a tubular continuous flow type reactor/heat exchanger which is heated externally in such a way that when the reaction mixture exits the last hot section of the reactor, at least the maximum temperature between 75° C. to 95° C. is achieved.

In one embodiment the rapid cooling is performed by supplying the cooling medium externally like chilled water supplied to reactor jacket or through chilled water or refrigerant supplied in coils attached to the reaction vessel.

In one embodiment the rapid cooling is done by adding ice, chilled water etc. directly to the reaction mixture.

In one embodiment the rapid cooling is done by passing the reaction mixture at or above 75° C. through a tubular continuous flow type reactor/heat exchanger which is cooled externally in such a way that when the reaction mixture exits the last cooled section of the reactor/heat exchanger, at least the temperature of 60° C. is achieved.

In still another embodiment, the present invention relates to a method of synthesis wherein the said ring closure is affected in presence of one or more than one oxidizing agent in catalytic or higher amounts.

In another embodiment, the DAPS synthesized in accordance to above described method can be purified by techniques selected from but not limited to filtration, recrystallization, organic solvent extraction and the like.

According to one embodiment of the present invention, the crude Methylene Blue obtained by the high purity process may contain various water insoluble impurities which can be easily removed by well-known filtration techniques. Further purification of crude Methylene Blue involves removal of precipitated water insoluble metal compounds (after the alkali treatment step) to obtain a metal free high purity Methylene Blue.

In one embodiment of the present invention, purification by filtration is carried out after ring closure and before rapid cooling.

In one embodiment of the present invention, purification by filtration is carried out after the rapid cooling.

In one embodiment of the present invention, purification by filtration is carried out after rapid cooling, maintaining the temperature of the reaction mixture in the range of 30 to 60° C.

In a preferred embodiment of the present invention, purification by filtration is carried out after rapid cooling, maintaining the temperature of the reaction mixture in the range of 30 to 60° C.

In one embodiment of the present invention, purification by filtration is carried out by filtering the hot aqueous reaction mixture through a centrifuge with a polypropylene bag as filter medium.

In one embodiment of the present invention, purification by filtration is carried out by filtering the hot aqueous reaction mixture through a bag filter with a polypropylene bag as filter medium.

In a preferred embodiment of the present invention, purification by filtration is carried out by filtering the hot aqueous reaction mixture through cascade of bag filters of decreasing pore size from 5 micron to 0.5 micron.

In one embodiment of the present invention, purification by filtration is carried out after ring closure and before rapid cooling by filtering the hot aqueous reaction mixture through a bag filter with a nylon bag as filter medium.

In further preferred embodiment of the present invention, purification by filtration is carried out after ring closure and before rapid cooling by filtering the hot aqueous reaction mixture through a centrifuge with a polypropylene bag as filter medium, followed by filtration through a polypropylene cartridge filter.

In a further preferred embodiment of the present invention, purification by filtration is carried out by using a cascade of polypropylene cartridge filters with stepwise decrease in the pore size of the cartridge filter.

In one preferred embodiment of the present invention, purification by filtration is carried out using four P.P cartridge filters in cascade each comprising pore size of 5 micron, 1 micron, 0.45 micron and 0.2 micron respectively.

In one embodiment of the present invention, purification by filtration is carried out by using 0.2 micron absolute filter as the terminal or final filter to remove bioburden, endotoxins and sub-micron precipitates.

In one embodiment of the present invention, purification by filtration is carried out by using diatomaceous earth based filter aid, before filtering through cartridge filter.

In one embodiment of the present invention, purification by filtration is carried out by using silica based filter aid.

In a highly preferred embodiment of the present invention, purification by filtration is carried out after the alkali treatment of crude methylene blue.

In one embodiment of the present invention, purification by recrystallization is carried out by dissolving the crude methylene blue in water by heating, acidifying and cooling.

In one embodiment of the present invention, purification by recrystallization is carried out by dissolving the crude methylene blue in distilled water, followed by warming till temperature is between 40° C. to 60° C., then acidification using acid selected from hydrochloric acid, sulphuric acid, acetic acid and the like, followed by cooling to temperature between 0° C. to 20° C. to effect crystallization.

In a preferred embodiment of the present invention, purification by recrystallization is carried out by dissolving the crude methylene blue in deionized water and warming the solution till temperature is between 40° C. to 50° C. to make a 3 to 5% concentration solution of crude methylene blue. After optional filtration, the solution is acidified using hydrochloric acid at temperature above 30° C. to bring the pH in the range of 1 to 1.5. The warm acidified solution is cooled to temperature below 20° C. to obtain the crystals of purified methylene blue.

In a more preferred embodiment, the crude methylene blue is dissolved in deionized water with warming the solution till 40° C. to make a 3% concentration solution of crude methylene blue. After filtration through a cascade of filter cartridges, the solution is acidified using hydrochloric acid at temperature between 35° C. to 40° C. to bring the pH to 1.0. The warm acidified solution is cooled to temperature 15° C. to obtain the crystals of purified methylene blue.

In a further preferred embodiment of the present invention, purification by recrystallization is carried out by dissolving the crude methylene blue in deionized water with warming the solution till 40° C. to make a 3% concentration solution of crude methylene blue. The solution is treated with alkali to bring the pH in the range of 8.0 to 9.0. After filtration through a cascade of filter cartridges comprising filters of pore size 5 micron, 1 micron, 0.5 micron and 0.2 micron, the solution is acidified using hydrochloric acid at temperature between 35° C. to 40° C. to bring the pH to 1.0. The warm acidified solution is cooled to temperature 15° C. to obtain the crystals of purified methylene blue.

In a highly preferred embodiment of the present invention, purification by recrystallization is carried out by dissolving the crude methylene blue in deionized water with warming the solution till 40° C. to make a 3% concentration solution of crude methylene blue. The solution is treated with sodium carbonate to bring the pH to 9.0. After filtration through a centrifuge followed by cascade of filter cartridges comprising filters of pore size 5 micron, 1 micron, 0.5 micron and 0.2 micron, the solution is acidified using hydrochloric acid at temperature between 35° C. to 40° C. to bring the pH to 1.0. The warm acidified solution is allowed to cool naturally to room temperature and thereafter forced cooled to temperature 10° C. to obtain the crystals of purified methylene blue which are centrifuged, washed with acidified deionized water and dried under vacuum.

In one embodiment of the present invention, purification by recrystallization is carried out by using the solvent selected from but not limited to a mixture of water and water soluble organic solvent selected from but not limited to methanol, ethanol, acetonitrile, acetone and the like. In a preferred embodiment, the water soluble solvent selected is acetonitrile.

In one embodiment of the present invention, purification by recrystallization is effected by adding a salt selected from but not limited to sodium chloride, potassium chloride, sodium sulphate and the like.

In one embodiment of the present invention, purification by recrystallization is effected by adding an anti-solvent to the aqueous solution selected from but not limited to acetone, ethanol and the like.

In one embodiment of the present invention, purification by recrystallization is carried out by acidification below the room temperature.

In one embodiment of the present invention, purification of crude methylene blue is carried out by organic extraction. The crude methylene blue is dissolved in water and extracted one or multiple times using a solvent selected from but not limited to chloroform, carbon tetrachloride, dichloromethane and the like. The separated aqueous layer is subjected to acidification or salting out to obtain purified methylene blue.

In a preferred embodiment of the present invention, purification of crude methylene blue by organic extraction is carried out wherein the aqueous solution of methylene blue of pH between 8.0 to 9.5 is subjected to multiple organic solvent extraction using chloroform or dichloromethane. The separated aqueous layer is subjected to acidification and cooling to obtain purified methylene blue crystals.

According to one embodiment, the present invention discloses a pharmaceutical composition comprising high purity methylene blue, prepared according to the method of present invention.

In another embodiment, the present invention discloses a pharmaceutical composition comprising high purity methylene blue, for treatment of methemoglobinemia, comprising an aqueous solution of 0.1% to 2% methylene blue, preferably 0.5% to 1% methylene blue.

In another embodiment, the present invention discloses a pharmaceutical composition comprising high purity methylene blue, in injectable form, for the treatment of methemoglobinemia, prepared by the steps of:
a) dissolving high purity methylene blue in injection grade water;
b) filtering the aqueous solution from a suitable filter to remove bacterial endotoxins; and
c) filling the filtered aqueous solution in vials in a sterile environment; wherein the content of methylene blue in the said composition is in between 0.1% to 2%, preferably 0.5% to 1%.

In another embodiment, the injectable 0.1% to 2% aqueous Methylene Blue composition is practically free from residual organic solvents and derivative compounds of methylene blue specifically phenothiazine ring nitrogen substituted derivatives.

According to an embodiment of the present invention, the pharmaceutical composition comprising high purity methylene blue is useful for treatment of methaemoglobinemia.

According to another embodiment of the present invention, the pharmaceutical composition comprising high purity methylene blue, is useful for treatment of any disease or condition, for prophylaxis of any disease or condition, for use as a diagnostic agent or aid, for use as a staining agent and for use as a photodynamic agent.

In one embodiment, the present invention relates to preparation of medicament comprising high purity Methylene Blue compound of Formula I.

In another embodiment, the present invention relates to use of high purity Methylene Blue compound of Formula I, for the preparation of medicament.

The high purity Methylene blue of the present invention can also be used to prepare medicaments for urinary infections, in the form of coated tablets comprising high purity methylene blue, other active ingredients and excipients.

The high purity Methylene blue of the present invention can also be used to prepare medicaments for urinary infections, in the form of coated tablets comprising high purity methylene blue, camphor monobromide and malva purpura.

The medicaments comprising high purity methylene blue can be prepared as tablets, or capsules.

In a preferred embodiment, the medicaments comprising methylene blue are prepared using high purity USP-42 quality or Ph Eur 9.0 quality methylene blue which is practically free from residual organic solvents.

In a highly preferred embodiment, the medicaments comprising methylene blue are prepared using a high purity USP-42 quality or Ph Eur 9.0 quality methylene blue which is practically free from residual organic solvents and practically free from derivative compounds of methylene blue, specifically those which are phenothiazine ring amine substituted derivatives such as acetyl-leucomethyleneblue, benzoyl-leucomethyleneblue, 3,7-di(dimethylamino)-10-acetyl-phenothiazine, etc.

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

The present invention is further explained in the form of following examples. However, it is to be understood that the following examples are merely illustrative and are not to be taken as limitations upon the scope of the invention.

The starting materials used in the synthetic procedures are described in Table 1. All starting materials are commercially available and were purchased before use.

TABLE 1
Starting materials and their source

| Material | BATCH No. | MAKE |
|---|---|---|
| Sodium Nitrite | J344D19 | RANKEM |
| Sodium Thiosulphate | 3163420119 | QUALIGENS |
| Aluminium Sulphate | 2992080218 | FISHER SCIENTIFIC |
| Sodium Dichromate | 2876270718 | QUALIGENS |
| Iron Filling | 256940018 | FISHER SCIENTIFIC |
| Copper Sulphate | N006D19 | RANKEM |
| Sulphuric Acid | 2730460418 | FISHER SCIENTIFIC |
| N,N-Dimethylaniline | R236A19 | RANKEM |
| Sodium Chloride | 3511020519 | QUALIGENS |
| Hydrochloric Acid | 3629300819 | QUALIGENS |

Preparation of Intermediates

Step 1: Preparation of p-Aminodimethylaniline

To a solution of 9.4 g of Sodium Nitrite, in 20 ml distilled water, was added 26 ml of sodium chloride solution (0.25 mg/ml), resulting in a solution of sodium nitrite (˜46 mL). 15 g N,N-Dimethylaniline and 18.3 g H₂SO₄ were taken separately.
In a 1 litre glass beaker, 15 g N,N-Dimethylaniline and 75 g ice were added followed by slow addition of 18.3 g of H₂SO₄ over 2 minutes. The beaker was cooled in an ice bath and sodium nitrite solution (prepared above) was added drop wise to it, with continuous stirring over a period of 5 hours. The temperature of the beaker was maintained between 0 to −3° C. After completion of addition of sodium nitrite, the solution was stirred additionally for 15 minutes followed by the addition of 13 ml H₂SO₄. The reaction mixture was stirred for 15 minutes and Iron fillings (50 g) were added to the reaction mixture over 3 minutes. The stirring was continued and the temperature rose to 40° C. in 15-20 minutes. Stirring was stopped and the reaction mixture was left standing for 36 hours, after which the reaction mixture was filtered through filter paper on Buchner funnel to remove the excess iron. The light brown filtrate obtained contains the required product p-Aminodimethylaniline in soluble acidic form.

Step 2: Preparation of Thiosulfonic Acid of Bindschedler's Green

To the solution of p-Aminodimethylaniline, obtained in Step 1, was added ice to bring the temperature to 0° C., and then H₂SO₄ (12.2 g) and 5 g ice were added. This was followed by the sequential addition of sodium dichromate (12.2 g in 30 ml water), aluminum sulfate hexadecahydrate (21.5 g in 100 ml water) and sodium thiosulfate pentahydrate (29.7 g in 60 ml water), in four equal portions in every 15 minutes, under stirring. The temperature of the reaction mixture was maintained at 0° C. and the reaction mixture was stirred for 1 hour. Dimethylaniline (13 g) was taken in 30 g ice and H₂SO₄ (12.5 g) was added to it. The dimethylaniline solution was then added to the reaction mixture under stirring followed by dropwise addition of Sodium dichromate solution (25.5 g in 40 ml water) to the reaction mixture in 1 hour, maintaining the temperature at 0° C. Another batch of sodium dichromate solution (6.8 g in 15 ml water) was added to the reaction mixture in 15 minutes, drop wise, maintaining the temperature at 0° C. using ice. The reaction mixture was stirred for another 30 minutes. The reaction mixture thus obtained is dark green in color and contains the zwitterionic indamine thiosulfonic acid of Bindschedler's green.

### Example 1: Conversion of the Thiosulfonic Acid of Bindschedler's Green to Methylene Blue by Ring Closure

The reaction mixture containing thiosulfonic acid of Bindschedler's green (obtained in Step 2) was warmed to 60° C. using a hot plate with stirring. After achieving temperature of 60° C., copper sulfate (1.8 g in 10 ml water) was added and the temperature was raised to 78° C. over a period of 10 minutes. At this stage, the color of the reaction mixture changes to blue, indicating the thiazine ring closure reaction. The reaction mixture was kept between 78 to 90° C. for 45 minutes, after which the beaker is removed from the hot plate.

Example 1A: Half portion of the reaction mixture prepared in Example 1 was immediately cooled to 77° C. over 5 minutes and was further cooled down to 60° C. in 20 minutes. This reaction mixture 1A was then filtered through filter paper using a Buchner funnel. The filtrate thus obtained was acidified to pH 1 using HCl. The acidified filtrate was left at ambient conditions for 18 hours and then kept in refrigerator for 6 hours for cooling to 5 to 8° C. The crystals of methylene blue were formed, which were filtered using a Buchner funnel and washed with 25 ml ice water acidified to 1 pH using HCl. The washed crystals were sucked dry and further dried at 50° C. for 5 hours in oven resulting in 5.4 g of desired product methylene blue.

Example 1B: The other half portion of the reaction mixture prepared in Example 1 was immediately cooled down to 77° C. in 1 minute and was further cooled down to 60° C. in 10 minutes. The reaction mixture 1B was filtered through filter paper using a Buchner funnel and the filtrate was acidified to pH of 1 using HCl. The acidified filtrate was left at ambient conditions for 18 hours and then kept in refrigerator for 6 hours for cooling to 5 to 8° C. The crystals of methylene blue were formed, which were filtered using a Buchner funnel and washed with 25 ml ice water acidified to 1 pH using HCl. The washed crystals were sucked dry and were further dried at 50° C. for 5 hours in oven resulting in 5.2 g of desired product methylene blue.

Example 2: The ring closure procedure for the conversion of the thiosulfonic acid of Bindschedler's Green to Methylene Blue was conducted with same materials and exactly in the same manner as Experiment 1 except that after achieving the temperature of 78° C., the reaction mixture was maintained between 78 to 90° C. for 25 minutes, followed by removing the beaker from the hot plate.

Example 2A: One half portion of the reaction mixture of Example 2 was immediately cooled to 77° C. in 5 minutes, followed by further cooling to 60° C. in 20 minutes. The reaction mixture 2A was filtered through filter paper using a Buchner funnel and the filtrate was acidified to pH 1 using HCl. The acidified filtrate was left at ambient conditions for 18 hours and then kept in refrigerator for 6 hours for cooling to 5 to 8° C. The crystals of methylene blue were formed, which were filtered using a Buchner funnel and washed with 25 ml ice water acidified to pH 1 using HCl. The washed crystals were sucked dry and were further dried at 50° C. for 5 hours in a laboratory oven to yield 5.1 g of desired product methylene blue.

Example 2B: The other half portion of the reaction mixture of Example 2 was immediately cooled to 78° C. in 1 minute, followed by further cooling to 60° C. in 10 minutes. The reaction mixture 2B was filtered through filter paper using a Buchner funnel and the filtrate was acidified to pH 1 using HCl. The acidified filtrate was left at ambient conditions for 18 hours and then kept in refrigerator for 6 hours for cooling to 5 to 8° C. The crystals of methylene blue formed were filtered using a Buchner funnel and washed with 25 ml ice water acidified to pH 1 using HCl. The washed crystals were sucked dry and were further dried at 50° C. for 5 hours in a laboratory oven to yield 5.6 g of desired product methylene blue.

Example 3: The ring closure procedure for the conversion of the thiosulfonic acid of Bindschedler's Green to Methylene Blue was conducted with same materials and exactly as the experiment 1 except that after achieving the temperature of 78° C., the reaction mixture was maintained between 78 to 90° C. for 10 minutes, followed by removing the beaker from the hot plate.

Example 3A: Half portion of the reaction mixture from Example 3 was taken immediately and cooled to 78° C. in 5 minutes followed by further cooled down to 60° C. in 20 minutes. The reaction mixture 3A was filtered through filter paper using a Buchner funnel and the filtrate was acidified to pH 1 using HCl. The acidified filtrate was left at ambient conditions for 18 hours and then kept in refrigerator for 6 hours for cooling to 5 to 8° C. The crystals of methylene blue were formed which were filtered using a Buchner funnel and washed with 25 ml ice water acidified to pH 1 using HCl. The washed crystals were sucked dry and further dried at 50° C. for 5 hours in a laboratory oven to yield 5.2 g of desired product methylene blue.

Example 3B: The other half portion of the reaction mixture from Example 3 was taken immediately and cooled down to 78° C. in 1 minute followed by further cooling to 60° C. in 10 minutes. The reaction mixture 3B was filtered through filter paper using a Buchner funnel and the filtrate was acidified to pH 1 using HCl. The acidified filtrate was left at ambient conditions for 18 hours and then kept in refrigerator for 6 hours for cooling to 5 to 8° C. The crystals of methylene blue were formed which were filtered using a Buchner funnel and washed with 25 ml ice water acidified to pH 1 using HCl. The washed crystals were sucked dry and were further dried at 50° C. for 5 hours in a laboratory oven to yield 4.9 g of desired product methylene blue.

Example 4: HPLC Analysis: The crude samples obtained from the examples 1A, 1B, 2A, 2B, 3A and 3C were analyzed using HPLC technique by following the method as mentioned in the European Pharmacopoeia 9.0 in the monograph of Methylene Blue by using similar column and same chromatographic conditions. The analysis was carried out to detect the percentage of various diaminophenothiazines (Methylene Blue, Azure B, Azure C and Azure A) in the products obtained from examples 1A, 1B, 2A, 2B, 3A and 3C. Since these were crude samples which were the product of the very first isolation of Methylene Blue, the process impurities other than diaminophenothiazines appearing in the HPLC chromatograms have not been considered and those were excluded while performing the integration on the chromatograms. The purpose of this analysis is to demonstrate that the diaminophenothiazines such as Azure B, Azure C, Azure A, etc which are formed as degradation impurities are substantially low in comparison to methylene blue, in the crude product itself which is not subject to any substantial purification. The results of the analysis are shown in Table 2.

TABLE 2
HPLC analysis results

| | Example | Methylene | Azure | Azure | Azure |
|---|---|---|---|---|---|
| Sample No | No | Blue % | B % | C % | A % |
| PAT01EXP01A | 01A | 97.86 | 2.13 | BDL | BDL |
| PAT01EXP01B | 01B | 98.10 | 1.89 | BDL | BDL |
| PAT01EXP02A | 02A | 98.03 | 1.96 | BDL | BDL |
| PAT01EXP02B | 02B | 98.12 | 1.88 | BDL | BDL |
| PAT01EXP03A | 03A | 98.42 | 1.57 | BDL | BDL |
| PAT01EXP03B | 03B | 98.39 | 1.60 | BDL | BDL |

*BDL: Below Detection limit as set by European Pharmacopoeia (limit of 0.05%)

All the crude samples were found to contain the Azure B well below 2.5% and other demethylation impurities such as Azure C & Azure A below the limit of 0.05% of European pharmacopoeia. The above results are of the samples which were not subjected to any of the well-known and easy purification techniques like recrystallization, alkali treatment, solvent extraction, etc. With the purification techniques well established and well known in the art, the diaminophenothiazines like Methylene Blue synthesized using the present invention can be easily purified further to even higher purities levels than the current specifications of international pharmacopoeias like USP and Ph. Eur. The low levels of Azure B and other impurities like Azure C & Azure A (levels of Azure B less than 2.5% and levels of Azure C and Azure A less than 0.1% and the other related diaminophenothiazine impurities well below the limits of USP & Ph. Eur) obtained in the samples prepared using the present invention were never obtained using any known and published methods without carrying out complicated purification techniques.

Experiment 5: Purification of Crude Methylene Blue Sample No. PAT01EXP01B

The crude sample (3 grams) obtained from experiment no. 1B (sample no. PATO1EXP01B) was weighed and dissolved in 100 ml of distilled water at 40° C. under stirring. The dissolution was complete in 15 minutes. The pH of the solution was adjusted to 8.5 using dilute sodium carbonate solution in distilled water, and the process was carried out slowly over 15 minutes. Stirring was continued for another 15 minutes and then the solution was filtered over Buchner funnel using a Whatman's filter paper. The filtrate was collected in a glass beaker and then acidified with hydrochloric acid to adjust pH to 1.0. The acidified filtrate was left in ambient conditions overnight and then cooled in refrigerator till 10° C. The crystals obtained were filtered over Buchner funnel and washed with chilled distilled water. The wet crystals were re-dissolved in 75 ml of distilled water at 40° C. under stirring and acidified with hydrochloric acid to pH 1.0. The acidified solution was left in ambient conditions overnight and then cooled in refrigerator till 10° C. The crystals obtained were filtered over Buchner funnel and washed with chilled distilled water. The crystals were dried in laboratory oven at 60° C. for 3 hours resulting in highly crystalline and pure Methylene Blue (2.2 grams).

Experiment 6: HPLC Analysis of the Purified Methylene Blue.

The sample of Methylene Blue crystals obtained in the Experiment 5 was analyzed by HPLC as per the method described in Example 4. The results are depicted in the below table.

| | Example | Methylene | | | | Other |
|---|---|---|---|---|---|---|
| Sample No | No | Blue % | Azure B % | Azure C % | Azure A % | Impurities |
| PAT01EXP01BRC | 5 | 98.01% | 1.98% | Not | Not | Not |
| | | | | Detected | Detected | Detected |

The HPLC analysis of Example 4 only focused on detecting quantities of unwanted impurities in the form of Azure A, Azure B and Azure C, whereas in the HPLC analysis of the sample (purified Methylene Blue as obtained in Example 5) as per the present example, all impurities in the chromatogram were taken into account. The presence of other impurities in addition to Azure A, Azure B and Azure C was also evaluated. The results of HPLC analysis indicate Azure B as the only impurity in the purified sample of Methylene Blue, and there were no other impurities detected (applying a disregard limit of 0.05% as per European Pharmacopoeia monograph of Methylene Blue), proving that the process of present invention indeed leads to synthesis of Methylene Blue of high purity as per the requirements of USP and EP.

Experiment 7: The process of preparation of Methylene blue was carried out exactly as per Example 1 and Example 1B, except that instead of copper sulphate that was used in Example 1, 2 grams of cupric chloride was used resulting in 11 grams of Methylene Blue.

Experiment 8: The process of preparation of Methylene blue was carried out exactly as per Example 3 and Example 3B to obtain the crude methylene blue, which was repurified. All procedures of purification of crude methylene blue were performed in glass apparatus cleaned with dilute aqua regia and rinsed with USP quality purified water. No metal contact was ensured all the time.

The crude methylene blue obtained was re-dissolved in 300 ml purified water (USP quality water with total organic carbon (TOC) less than 50 ppb). The solution was stirred and warmed till 45° C., stirring for 15 minutes at 45° C. for complete dissolution. The solution was treated under stirring with sodium carbonate solution to bring the pH level to 8.8. The alkaline solution was filtered using a Buchner funnel installed with filter paper. The filtrate was taken and the pH of the warm filtrate was adjusted to 1.0 using high purity hydrochloric acid (with low trace metal content). The acidified warm filtrate was left overnight for cooling to room temperature. Thereafter it was cooled in a refrigerator to 10° C. Needle shaped crystals were formed, which were filtered over a Buchner funnel and washed with chilled acidified water (using HCl) of pH 1.0. The wet crystals were re-dissolved in purified water, treated with sodium carbonate, filtered and recrystallized, exactly as per the above procedure, three times. The resulting highly crystalline, needle shaped and lustrous Methylene Blue crystals (4.9 gms) were were dried in a laboratory oven for 3 hours at 60° C. to obtain 4.0 gins of highly pure dried crystals of Methylene blue. The methylene blue thus obtained was analyzed for sulphated ash content as per the European Pharmacopoeia 9.0 method and the sulphated ash content was found to be 000. The obtained methylene blue was also analyzed for metals impurities using ICP-OES technique for metals as per the European Pharmacopoeia 9.0 monograph and none of the metal was found higher than 0.5 times the limit prescribed in the said monograph. The results are depicted in the below table.

| | Limit as per Ph | |
|---|---|---|
| Element Name | Eur and USP (ppm) | Results |
| Arsenic* | 8 ppm | Less than 1 ppm |
| Zinc | 100 | Less than 50 ppm |
| Copper* | 200 | Less than 10 ppm |
| Aluminum | 100 | Less than 50 ppm |
| Cadmium | 1 | Not Detected |
| Chromium | 100 | Less than 50 ppm |
| Tin | 10 | Less than 2 ppm |
| Iron | 200 | Less than 50 ppm |
| Manganese | 10 | Less than 2 ppm |
| Mercury | 1 | Less than 0.2 ppm |
| Molybdenum | 10 | Less than 2 ppm |
| Nickel | 10 | Less than 2 ppm |
| Lead | 10 | Less than 1 ppm |

*Limit of Arsenic and copper are as per USP-42 monograph and rest all are as per Ph Eur 9.0 monograph.

Experiment 9: The procedure as described in Experiment 1 was conducted till the formation of thiosulfonic acid of bindschedler's green. Thereafter, the suspended solids in reaction mixture were isolated over a buchner funnel. The isolated greenish cake contained the thiosulfonic acid of bindschedler's green, precipitated chrome and other impurities and the cake was not subjected to any purification. 500 ml distilled water was taken in glass beaker and acidified to pH 2.0 using HCl. The beaker was placed on a hot plate with magnetic stirrer for heating. At temperature of 75° C., 2 grams of copper sulphate was added and when temperature reached 95° C., the isolated cake containing bindschedler's green was added at once under rigorous stirring. The reaction mixture showed deep blue color within 1 minute after addition. Stirring was continued for another 10 minutes and after that the stirring was stopped and the glass beaker removed from hot plate. The beaker was placed in a water bath for fast cooling. In 10 minutes, the temperature was 58° C. The hot filtrate was filtered over a Buchner funnel installed with filter paper. The warm filtrate was acidified using HCl to bring the pH to 1.0 and left for cooling overnight at room temperature. The greenish crystals of Methylene Blue were formed, which were isolated by filtration over a Buchner funnel, washed with chilled water and dried in lab oven at 60° C. for 8 hours resulting in 12.4 gm of Methylene Blue. The HPLC analysis of the Methylene Blue thus obtained was done as per the Ph. Eur. 9.0 chromatographic conditions. The percentage of impurity as compared to methylene blue was calculated using below formula:

$$\% impurity = 100 * [ ( peak area of impurity ) / ( peak area of Methylene Blue ) ]$$

Impurities other than Azure B, Azure C and Azure A were not considered for integration in this crude sample.

| Impurity name | Amount detected by HPLC |
|---|---|
| Azure B | 1.6% (of methylene blue) |
| Azure A | Below disregard limit |
| Azure C | Below disregard limit |

Disregard limit of 0.03% of peak area of methylene blue was applied in the above analysis.

The Azure B levels obtained in this sample are much lower than those achievable through the process described in U.S. Pat. No. 7,790,881 wherein Cr(VI) mediated oxidative coupling, Cr(VI) reduction and isolation and purification of thiosulfonic acid of bindschedler's green is performed and methylene blue is synthesized without organic extraction. The Example 1 of U.S. Pat. No. 7,790,881 discloses the results of sample CM-pd-378, wherein 2.89% Azure B has been reported. Consequently, it is clear that the novel and inventive process of the present invention wherein rapid heating and rapid cooling are carried out during cyclization step, and product formed is exposed for a very limited time to higher temperatures, leads to an evident improvement in the purity of product obtained, i.e., methylene blue wherein the quantity of unwanted impurity Azure B is drastically reduced.

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### ADVANTAGES OF THE PRESENT INVENTION

The present invention provides an improved method of synthesizing high purity diaminophenothiazine compounds, specifically Methylene Blue, that satisfies the existing needs, as well as others, and generally overcomes the deficiencies found in the prior art.

The present invention provides an improved method of synthesizing high purity diaminophenothiazine compounds, more specifically Methylene Blue, without the presence of unwanted impurities.

The present invention provides a method of synthesizing high purity Methylene Blue, with purity parameters which can meet the limits of degradation impurities mainly Azure B as per the requirements of the international pharmacopoeias like USP and EP.

The present invention provides a high purity process for the synthesis of Methylene Blue which is commercially viable and safe on industrial scale.

The present invention provides an improved method of synthesizing high purity Methylene Blue as per pharmaceutical grade, i.e., compound having purity safe for human consumption.

The present invention provides a method of synthesizing high purity Methylene Blue, which does not involve use of organic solvents.

The present invention provides a method of synthesizing high purity Methylene Blue that provides the desired product practically free from the organic solvents, or contains lesser than the allowed residual solvents limits of the different pharmacopoeias.

The present invention provides an improved method of synthesizing high purity Methylene Blue, which is practically free from the reduced leuco and other substituted forms.

The present invention provides a method of synthesizing high purity Methylene Blue comprising low Azure B impurity content, without the need of isolation and purification of the thiosulfonic acid of bindschedler's green intermediate.

The present invention provides a method of synthesizing high purity Methylene Blue without the need to derivatize methylene blue at the ring nitrogen of phenothiazine ring.

1-39. (canceled)

40. A method of synthesizing high purity Methylene Blue of Formula I and its pharmaceutically acceptable salts and hydrates thereof, from compound of Formula II, said method comprising the following steps: 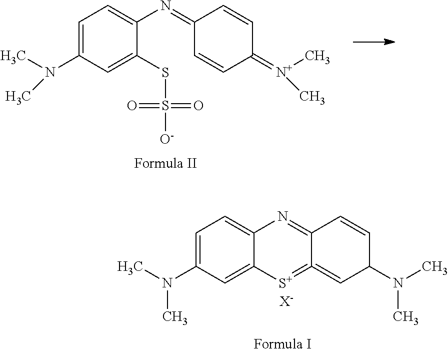
a) Subjecting the zwitterionic indamine compound (ZIC) represented by Formula II, to rapid heating in a liquid medium to achieve a maximum temperature which is at least the temperature required for thiazine ring cyclization of the ZIC to form diaminophenothiazine salt of Formula I (DAPS),
wherein said thiazine ring cyclization is performed in presence of at least one of a copper source, wherein the copper source is selected from copper sulphate, copper compound and copper ions used in quantities ranging from (on anhydrous basis) 0.05 mol per mole of ZIC to 0.5 mol per mole of ZIC, wherein the liquid medium is aqueous medium having pH in the range of range of 0.5 to 6.5,
wherein the temperature required for the thiazine ring closure is in the range of 70-95° C. and wherein ZIC of Formula II is subjected to the rapid heating such that the rise of temperature from 60° C. to 78° C. is achieved in 1 to 15 minutes,
b) exposing the cyclized DAPS of Formula I to elevated temperatures for very short period of time such that the exposure of cyclized DAPS to temperatures in the range of 78-100° C. for 1 to 15 minutes;
C) rapid cooling the reaction mixture containing the DAPS, so as to allow the exposure of the ZIC and/or the DAPS to elevated temperatures for very short amount of time, wherein the rapid cooling is carried out such that the temperature is brought down up to 60° C. in 1 to 15 minutes; and
d) after performing the ring closure, isolating the product formed, DAPS;
wherein X⁻ is chloride or any other counter anion to achieve electrical neutrality.

41. The method as claimed in claim 40, wherein the prepared high purity Methylene Blue comprises:
a) Azure B less than 3% as per European Pharmacopoeia 9.0 HPLC method;
b) Azure A less than 0.1% as per European Pharmacopoeia 9.0 HPLC method;
c) Azure C less than 0.1% as per European Pharmacopoeia 9.0 HPLC method;
d) Sulphated Ash less than 0.25% as per European Pharmacopoeia 9.0 method.

42. The method as claimed in claim 40, wherein the prepared high purity Methylene Blue comprises:
a) Azure B less than 3% as per European Pharmacopoeia 9.0 HPLC method;
b) Azure A less than 0.1% as per European Pharmacopoeia 9.0 HPLC method;
c) Azure C less than 0.1% as per European Pharmacopoeia 9.0 HPLC method;
d) Sulphated Ash less than 0.25% as per European Pharmacopoeia 9.0 method;
e) Less than 100 ppm of total residual organic solvent content of one or more solvents selected from methanol, ethanol, acetonitrile, acetone, ethyl acetate, dimethylformamide, acetic acid, dichloromethane, carbon tetrachloride, chloroform, cyclohexane, diethyl ether, dimethyl sulfoxide, dichloroethane, 1-propanol, 2-propanol, toluene, tetrahydrofuran or any class 1, class 2 or class 3 solvent, as mentioned in ICH Q3C(R6) guideline and measured by a gas chromatograph with FID detector or a mass detector.

43. The method as claimed in claim 40, wherein total residual organic solvent content of one or more solvents is less than 50 ppm, preferably less than 10 ppm, more preferably less than 5 ppm, highly preferably less than 1 ppm and most preferably less than 0.1 ppm and the content of elemental impurities in high purity Methylene Blue is less than the amount allowable for each elemental impurity according to limits of European Pharmacopoeia 9.0.

44. The method as claimed in claim 40, wherein content of Azure B in high purity Methylene blue is less than 2.5%, preferably less than 2.0%, more preferably less than 1.8% as per European Pharmacopoeia 9.0 HPLC method.

45. The method as claimed in claim 40, wherein the content of elemental impurities in high purity Methylene Blue is less than the amount allowable for each elemental impurity according to limits of European Pharmacopoeia 9.0, preferably less than 0.75 times the limit prescribed in the European Pharmacopoeia 9.0 and more preferably less than 0.5 times the limit prescribed in the European Pharmacopoeia 9.0.

46. The method as claimed in claim 40, wherein content of Azure A in high purity Methylene blue is less than 0.05% and content of Azure C is less than 0.05%, as per European Pharmacopoeia 9.0 HPLC method.

47. The method as claimed in claim 40, wherein content of MVB is less than 0.05%, content of Thionin is less than 0.05%, content of any diaminophenothiazine compound other than methylene blue and Azure B is less than 0.05%.

48. The method as claimed in claim 40, wherein the isolation step of DAPS optionally involve additional steps including, filtration, pH adjustment, acidification, salt formation, crystallization, cooling, salting out and precipitation.

49. The method as claimed in claim 40, wherein one or more treatment steps can be optionally applied after the ring closure and before the isolation of DAPS, like pH adjustment, alkalization, in order to remove inorganic and other impurities.

50. The method as claimed in claim 40, wherein DAPS is isolated by salt formation, preferably as corresponding chloride salt or zinc chloride salt.

51. The method as claimed in claim 40, wherein the DAPS formed after the ring closure is isolated after its synthesis from the reaction mixture in which it was synthesized and optionally purified.

52. The method as claimed in claim 40, wherein high purity Methylene Blue is further derivatized into reduced and stable forms selected from acetyl leuco-Methylene Blue, Benzoyl Leuco-methylene Blue, Leuco-methylene Blue Dihydrochloride, Leuco-methylene Blue Mesylate and Leuco-methylene Blue Ascorbate, using reducing agents.

53. The method as claimed in claim 40, wherein the DAPS synthesized is further purified by techniques selected from filtration, alkalization, recrystallization and organic solvent extraction.

54. The method as claimed in claim 40, wherein no organic solvent selected from methanol, ethanol, acetonitrile, acetone, ethyl acetate, dimethylformamide, acetic acid, dichloromethane, carbon tetrachloride, chloroform, cyclohexane, diethyl ether, dimethyl sulfoxide, dichloroethane, 1-propanol, 2-propanol, toluene, tetrahydrofuran or any class 1, class 2 or class 3 solvent mentioned in ICH Q3C(R6) guideline, is used in the synthesis, purification or isolation of final purified methylene blue.

55. The method as claimed in claim 40, wherein the cyclized DAPS is not exposed to temperatures above 60° C. for more than 30 minutes or more preferably 15 minutes.

56. The method as claimed in claim 40, wherein the said ZIC in isolated or mixture form, is added to the said liquid medium, at temperatures higher than 60° C., preferably 75° C., more preferably 85° C.

57. A high purity and practically organic solvent free Methylene Blue compound of Formula I and its pharmaceutically acceptable salts and hydrates thereof; wherein the high purity is characterized by following:
a) Azure B less than 3% as per European Pharmacopoeia 9.0 HPLC method;
b) Azure A less than 0.1% as per European Pharmacopoeia 9.0 HPLC method;
c) Azure C less than 0.1% as per European Pharmacopoeia 9.0 HPLC method;
d) Sulphated Ash less than 0.25% as per European Pharmacopoeia 9.0 method;
e) Less than 100 ppm of total residual organic solvent content;
wherein X⁻ is chloride or any other counter anion to achieve electrical neutrality.

58. A high purity Methylene Blue according to claim 57, wherein the total residual organic solvent content is less than 50 ppm.

59. A high purity Methylene Blue according to claim 57, wherein the total residual organic solvent content is less than 10 ppm.

60. A high purity Methylene Blue according to claim 57, wherein the total residual organic solvent content is less than 1 ppm.

61. A high purity Methylene Blue according to claim 57, wherein there are no detectable traces of any residual organic solvent.

62. A high purity Methylene Blue according to claim 57, wherein the residual organic solvents are selected from methanol, ethanol, acetonitrile, acetone, ethyl acetate, dimethylformamide, acetic acid, dichloromethane, carbon tetrachloride, chloroform, cyclohexane, diethyl ether, dimethyl sulfoxide, dichloroethane, 1-propanol, 2-propanol, toluene, tetrahydrofuran or any class 1, class 2 or class 3 solvent, as mentioned in ICH Q3C(R6) guideline.

63. A high purity Methylene Blue according to claim 57, wherein the said methylene blue compound is additionally free of any phenothiazine ring nitrogen derivatized compound.

64. A high purity Methylene Blue according to claim 57, wherein additionally the content of elemental impurities in high purity Methylene Blue is less than the amount allowable for each impurity according to limits of European Pharmacopoeia 9.0.

65. A high purity Methylene Blue according to claim 57, wherein additionally the content of elemental impurities in high purity Methylene Blue is less than 0.75 times the amount allowable for each impurity according to limits of European Pharmacopoeia 9.0.

66. A high purity Methylene Blue according to claim 57, wherein additionally the content of elemental impurities in high purity Methylene Blue is less than 0.5 times the amount allowable for each impurity according to limits of European Pharmacopoeia 9.0.

67. A high purity Methylene Blue according to claim 57, wherein content of Azure B in high purity Methylene blue is less than 2.5%, preferably less than 2.0%, more preferably less than 1.8% as per European Pharmacopoeia 9.0 HPLC method.

68. A high purity Methylene Blue according to claim 57, wherein content of Azure C & Azure A in high purity Methylene blue is less than 0.05% as per European Pharmacopoeia 9.0 HPLC method.
